(19) United States
(12) Patent Application Publication
(10) Pub. No.: US 2007/0142629 A1
(43) Pub. Date: Jun. 21, 2007

(54) Multichemistry fractionation

(75) Inventors: Luc Guerrier, Versailles (FR); Egisto Boschetti, Croissy sur Seine (FR); Frederic Fortis, Cergy (FR)

Correspondence Address: CIPHERGEN c/o FOLEY & LARDNER LLP, 3000 K STREET NW, SUITE 500, WASHINGTON, DC, 20007, US

(73) Assignee: Ciphergen Biosystems, Inc.

(21) Appl. No.: 10/558,649

(22) PCT Filed: Jun. 16, 2005
(86) PCT No.: PCT/US05/21489

§ 371 Date: Nov. 30, 2005

Related U.S. Application Data
(60) Provisional application No. 60/580,627, filed on Jun. 16, 2004.
(60) Provisional application No. 60/591,319, filed on Jul. 27, 2004.

(51) Int. Cl. C07K 1/12

(52) U.S. Cl. 530/412

(57) ABSTRACT

Methods, apparatuses, and kits for fractionating complex mixtures of biological molecules are provided. In one aspect the methods provided include providing a series of different sorbents, introducing the complex mixture to the series of sorbents, contacting serially the complex mixture with each of the sorbents, and capturing biomolecular components from the complex mixture on the sorbents so that each of the sorbents captures a substantially unique subset of said plurality of biomolecular components.

This application claims the priority benefit of U.S. Provisional Application No. 60/591,319 filed on Jul. 27, 2004 and U.S. Provisional Application No. 60/580,627, filed on Jun. 16, 2004, both of which are hereby incorporated herein by reference.

### BACKGROUND

The present invention relates generally to the fields of protein chemistry and analytical chemistry, and, more particularly, to the purification of proteins and other chemicals of biological origin from complex mixtures of such chemicals. The invention has applications in the areas of protein chemistry, analytical chemistry, clinical chemistry, drug discovery, and diagnostics.

The analysis of the protein content from a tissue extract or biological liquid provides a very elegant and powerful method for understanding the phenotypic state of an organism. A comparison of the differences between the protein content of a phenotypically “standard” or “normal” sample and a non-standard sample provide a means to identify pathological phenotypes and, possibly, identify palliative or curative treatments. Thus, in principal, the analysis of protein content in tissues and other biological samples has great potential to provide fast, accurate diagnoses and better treatments for diseases.

However, the detection and quantitation of individual peptides or proteins (or other molecules of biological origin) in a complex sample is not straightforward, given the large dynamic range of concentrations of molecular species in a typical sample (˜10⁸). In other words, the most common molecular species is present in an amount that is on the order of one hundred million-time greater than the least common molecular species in a given sample volume. Current materials and methods for isolating and quantifying the species in a given biological sample simply are not sufficient to isolate reliably all of the components of such a mixture. Typically, the dominant molecular species will mask those species present in concentrations less than about one one thousandth of the dominant species. For biological samples, such as blood, alubmin and immunoglobulins are two of the most the predominant molecular species; and attempts to identify various enzymes, antibodies, proteins, or secondary metabolites that may have relevance as disease markers, or which may be relevant for drug discovery, are complicated by these hordes that limit the resolving power, sensitivity, and loading capacity of the two most commonly used analytical techniques: 2-dimensional electrophoresis (2DE) and mass spectrometry (MS). For example, the presence of such highly abundant proteins in a sample produces large signals with consequent signal overlap (in 2DE) or signal suppression (in MS) of the other species present in the sample, which, complicates analysis and undermines any conclusions about the catalog of molecular species present in the sample.

Classical approaches to addressing these complications have consisted in separating proteins that are very concentrated, or in reducing the complexity of the entire mixture by various fractionation methods. Such methods have included: sub-cellular fractionation (Lopez, M. F., Electrophoresis, 2000, 21:1082-1093; Hochstrasser, D. F., et al, Electrophoresis, 2000, 21:1104-1115; Dreger, M., Mass. Spectrmetry Reviews, 2003, 22:27-56; Patton, W. F., J. Chromatography B, 1999, 722:203-223; Mc Donald T. G et al, Basic Res. Cardiol., 2003, 98:219-227; Patton, W. F., et al, Electrophoresis, 2001, 22:950-959; Gemer C., et al, Mol. & Cellular Proteomics, 2002, 7:528-537), isoelectric separation (Issaq, J. H., et al, Electrophoresis, 2002, 23:3048-3061; Dreger, 2003; Righetti P. G., et al, J. Proteome Res., 2003: 2, 303-311; Righetti P. G., et al, Electrophoresis, 2000: 21, 3639-3648; Rossier J. S., et al., Electrophoresis, 2003: 24, 3-11; Faupel M., et al, Proteomics, 2002, 2:151-156; Miller B. S., et al, Electrophoresis, 2003, 24:3484-3492;), mono-dimensional SDS-electrophoresis (Issaq, J. H., et al 2002,7,15), molecular sizing (Issac, J. H., et al. 2003, Hochstrasser, et al. 2000) and liquid chromatography (Issaq, J. H., et al 2002, Hochstrasser, et al. 2000) are common ways to proceed prior to 2DE or directly to MS or LC-MS identification. For example ICAT methodology involves an avidin-affinity separation of biotinylated tagged trypsic peptides (Issaq, J. H., et al 2002, Hochstrasser, et al. 2000; Moseley, A. M., Trends in Biotechnology, 2001, 19:S10). Other fractionation methods use ion exchange (Lopez, M. F., 2000,17), IMAC for calcium binding protein (Lopez, M. F., et al, Electrophoresis, 2000, 21:3427-3440) or phospho-proteins (Hunt, D. F., et al, Nat. Biotechnol., 2002, 20:301-305), hydrophobic (Lopez, 2000), heparine (Hochstrasser, et al. 2000) or lectin (Hochstrasser, et al. 2000; Lopez, 2000; Regnier, F., et al, J. Chromatography B, 2001, 752:293-306) affinity chromatography to get the protein sample less complex. Two-dimensional liquid chromatography used for intact protein fractionation or their trypsic digests, generally uses RP for the second dimension, combined with ion exchange (Yates, J. R., Nature Biotech., 1999, 17:676-682, Unger, K. K., et al, Anal. Chem., 2002,74:809-820), chromato-focusing (Wall, D., et al, Anal. Chem., 2000, 72:1099-1111), size exclusion (Opiteck, G., Anal. Biochem., 1998, 258:349-361), affinity (Regnier 2001), or another RP (Chicz R., et al, Rapid Commun. in Mass Spectrometry, 2003, 17:909-916) as the first chromatography step. Multidimensional chromatography in proteomic fractionation generally never exceed two dimensions due to high number of fractions to manage (pH-adjustment, desalting, re-injection in second dimension) and analyze, especially when a tedious analytical methods as 2DE makes the final bottleneck.

Still there remains a pressing need to provide methods, materials, and apparatus for more efficient and more reliable separation of samples containing complex mixtures of biological substances. The present invention meets these and other needs.

### SUMMARY

The present invention addresses these and other needs by providing methods, apparatuses, and kits that allow more efficient and reliable purification of complex mixtures of biological substance, especially proteins. The methods, apparatuses, and kits provided by the invention can be used in conjunction with additional purification and analytical techniques to identify and quantify the biological substances present in a given sample, especially proteins. Thus, the methods, apparatuses, and kits of the invention have important applications to proteomics, diagnostics, and drug discovery among other fields.

In one embodiment, the invention relates to methods for prefractionating a complex mixture including a plurality of different biomolecular components. One particular embodiment of the methods provided by the invention include providing a series of different sorbents, introducing the complex mixture to the series of sorbents, contacting serially the complex mixture with each of the sorbents, and capturing biomolecular components from the complex mixture on the sorbents so that each of the sorbents captures a substantially unique subset of said plurality of biomolecular components. In a more specific embodiment of the method, the method includes contacting the complex mixture with at least two different sorbents having different specificities including sorbents having high specificity, moderate specificity, and low specificity. A still more specific embodiment of the method includes selecting the sorbents to effect substantially complete capture of all biomolecular components from the complex mixture.

In one aspect, there is provided a method comprising providing a series of at least three different sorbents arranged in a progression of decreasing specificity; introducing a complex mixture to said series of sorbents; contacting serially said complex mixture with each of said sorbents; and capturing biomolecular components from said complex mixture on said sorbents, wherein each of said sorbents captures a substantially unique subset of said plurality of biomolecular components.

In another aspect, the invention provides an apparatus for prefractionating a complex mixture including a plurality of biomolecular components. In one embodiment, the apparatus of the invention includes a plurality of sorbents characterized by different adsorption specificities for different biomolecular component types coupled in a series arrangement. The sorbents are arranged such that introduction and passage of a buffered solution including (i) the complex mixture and (ii) a buffer that is compatible with the sorbents serially through the series arrangement of sorbents is effective to remove at least a portion of the mixture components from the mixture components from. In a more particular embodiment, the sorbents are arranged to define a progression in affinities for at least one biomolecular component type. In a more specific embodiment, the apparatus defines a substantially contiguous component-sequestering body. In a still more specific embodiment, the apparatus defines a substantially linear progression of adsorption specificities for at least one of the biomolecular component types.

In one example, there is provided an apparatus comprising at least three sorbents characterized by different adsorption specificities for different biomolecular component types coupled in a serial arrangement of decreasing specificity. In another, an apparatus can comprise in sequence: (a) a high specificity sorbent, (b) a moderate specificity sorbent, and (c) a low specificity sorbent, and said sorbents being coupled in a serial arrangement whereupon introduction and passage of a buffered solution including (i) a complex mixture and (ii) a buffer that is compatible with said materials serially through said serial arrangement of said materials is effective to remove substantially all of said biomolecular components from said complex mixture.

In still another aspect, the invention provides a kit for preparing an apparatus for prefractionating a complex mixture including a plurality of biomolecular components. In one embodiment, the kit provided by the invention includes a plurality of sorbents characterized by different adsorption specificities for different biomolecular component types and a compatible buffer chosen such that when the materials are coupled in a series arrangement, introduction and serial passage of a buffered solution including (i) the complex mixture and (ii) the buffer through the series arrangement of materials is effective to capture substantially all of the plurality of biomolecular components from the complex mixture.

In further embodiments, the biomolecular components isolated using the methods, apparatuses, and kits of the invention are eluted from the sorbents, for example, by at least one sorbent to water, a chaotropic agent, a lyotropic agent, an organic solvent, a change in ionic strength, a change in pH, a change temperature, a change pressure, or a combination of thereof. The isolated components can then be detected and identified using methods such as mass spectrometry, mono- and multi-dimensional gel electrophoresis, fluorimetric methods, high-pressure liquid chromatography, medium-pressure liquid chromatography.

These and other aspects and advantages of the invention will be more apparent when the description below is read with the accompanying figures.

### BRIEF DESCRIPTION OF THE DRAWINGS

FIG. 1 illustrates an embodiment of the method of the invention.

FIG. 2 illustrates the reduction in dynamic range of a sample, and the capture of the biomolecular components in the sample, by serial passage of the sample over successive sorbents ranging from sorbents having high specificity for abundant biomolecular species though sorbents having low specificity for any particular biomolecular species, according to one embodiment of the invention.

FIG. 3 is a graph comparing the fractionation method of the invention with other fractionation methods.

FIG. 4 is a graph of the results of the experiment described in Example 2 showing the superior resolving capabilities of the invention. Using the method of the invention, a sample spiked with insulin was detected on a specific sorbent chemistry (MEP-HYPERCEL, column A). In contrast, using prior art methods, insulin was detected in most of elution fractions from Q-HYPER-D with an undesirable signal dilution due to this spreading (column B).

FIG. 5 is a graph of the results of the experiment described in Example 2 showing the superior resolving capabilities of the invention. The ability of the method of the invention to capture insulin on a specific sorbent chemistry provides detection at concentrations as low as 1 fMol/μL in human serum (column A). Using prior art, single-chemistry, fractionation methods (Q-HyperD), a 2-log reduction in sensitivity was observed (100 fMol/μL, column B).

FIG. 6 is a mass spectrograph providing SELDI MS data obtained using a ProteinChip® Array CM10. “a”: initial serum proteins; “b”: C2 column; “c”: C4 column; “d”: C8 column. Molecular weight range explored is 2000-10000 Da.

FIG. 7 is a mass spectrograph providing SELDI MS data obtained using a ProteinChip® Array Q10. “a”: initial serum proteins; “b”: C2 column; “c”: C4 column; “d”: C8 column. Molecular weight range explored is 1000-6000 Da.

FIG. 8 provides SDS PAGE analysis of protein fractions under reduced conditions. “a” represents proteins stained after migration with Coomassie blue; “b” represents fraction eluted from C3, C4, C6 and FT (flowthrough), using a silver staining.

FIG. 9 is a mass spectrograph providing SELDI MS analysis of protein fractions eluted from C1, C2, C3, C4, C6 and FT (flowthrough), using a Q10 ProteinChip Array using a physiological buffer containing 2M urea

FIG. 10 is a mass spectrograph providing SELDI MS analysis of protein fractions eluted from C1, C2, C3, C4, C6 and FT (flowthrough), using a CM10 ProteinChip Array using a physiological buffer containing 2M urea.

### DETAILED DESCRIPTION

The present invention provides methods and systems for reducing the complexity of complex mixtures containing biomolecular components, i.e., chemical species generated by biological processes such as, but strictly limited to: proteins, nucleic acids, lipids, and metabolites. The methods and systems provided by the present invention allow isolation and detection of biomolecular components with greater sensitivity and efficiency that heretofore possible.

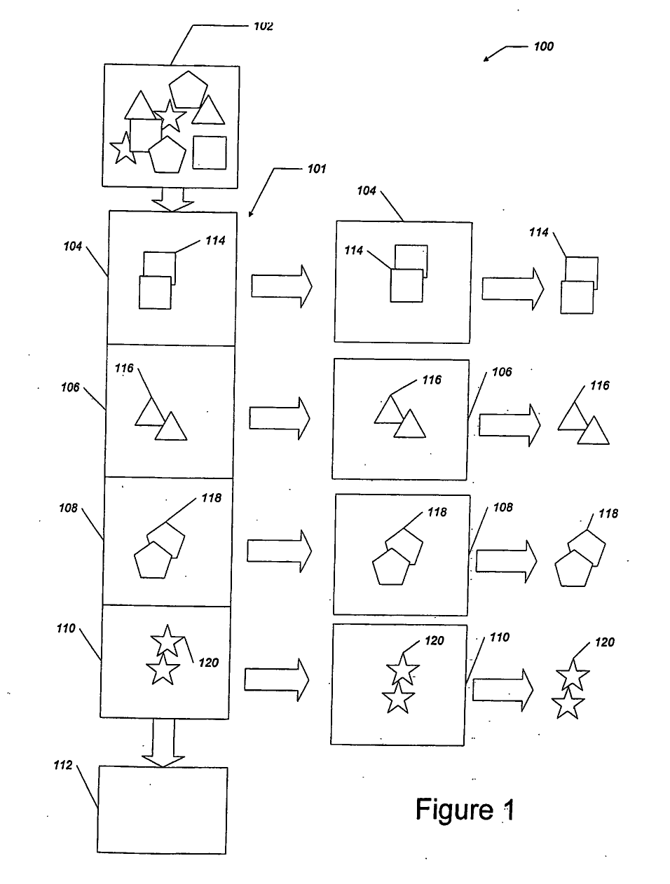

FIG. 1 provides an illustration of one embodiment of invention at 100. A sample solution containing a complex mixture including a plurality of different biomolecular components 101 is introduced to a sample fractionation column 102 for at least partial resolution as described hereinbelow. Column 102 includes a plurality of sorbent materials 104, 106, 108, and 110 arranged serially and through which solution 101 is passed to contact serially thereby each of the sorbent materials after which any remaining solution is eluted to a receptacle 112.

In one embodiment of the invention, the sorbent materials are chosen such that substantially all of the biomolecular components are captured by sorbents 104-110. In a more particular embodiment of the present invention, each of the sorbents 104-110 captures a substantially unique subset of the plurality of biomolecular components. Thus, sorbent 104 is effective to capture subset 114, sorbent 106 is effective to capture subset 116, sorbent 108 is effective to capture subset 118, and sorbent 110 is effective to capture subset 120. Following capture of the various subsets of the plurality of biomolecular components 101, the sorbents, including the captured biomolecular components, are isolated (i.e., removed from the column); and the subset components are eluted or otherwise removed from the sorbents for further processing as discussed in greater detail below.

As used herein “capture” refers to the ability of a sorbent to attract and reversibly retain one or more biomolecular components in solution 101 such that certain subsets of the biomolecular components are substantially completely removed from solution 101 during passage through column 102. Those of skill in the art of separating mixtures of chemicals of biological origin, such as protein purification, will appreciate that a sorbent's ability to retain a biomolecular component inherently includes a specificity of the sorbent for certain biomolecular components that is defined by the interaction between the sorbent and a biomolecular component under the ambient conditions in which the sorbent and the solution are in contact (e.g., the temperature and ionic strength or pH of the solution being passed through the column). The interaction can be any physicochemical interaction known or believed to be sufficient to cause sorption of a biomolecular component (or subset of biomolecular components) by the sorbent to substantially completely deplete the solution of the biomolecular component (or subset), but still allow subsequent elution of the captured biomolecular component(s).

Typical sorbent-biomolecular component interactions include without limitation: ion exchange (cation or anion); hydrophobic interactions; biological affinity (including interactions between dyes and ligands with proteins, or lectins with glycoconjugates, glycans, glycopeptides, polysaccharides, and other cell components); immunoaffinity (i.e., antigen-antibody interactions or interactions between fragments thereof); metal-chelate or metal-ion interactions, interactions between proteins and thiophilic materials, interactions between proteins and hydroxyapatite, and size exclusion. Many such materials are known to those having skill in the art of protein or nucleic acid purification. These materials can be made using known techniques and materials or purchased commercially. Descriptions of these materials and examples of methods for making them are described in Protein Purification Protocols 2^{nd} Edition, Cutler, Ed. Humana Press 2004, which is incorporated herein by reference in its entirety for all purposes.

Ion exchanging materials include strong and weak cation- and anion exchange resins. Strong cation exchanging ligands include sulfopropyl (SP) and methyl sulfonate (S). Weak cation exchange ligands include carboxymethyl (CM). Strong anion exchange ligands include quaternary ammonium and quaternary aminoethyl (QAE). Weak anion exchange ligands include diethylaminoethyl (DEAE). Examples of suitable ion-exchange materials include without limitation, the materials sold commercially under the trade names: Q-, S-, DEAE- and CM CERAMIC HYPERD®; DEAE-, CM-, and SP TRISACRYL®; M-, LS-; DEAE-, and SP SPHERODEX® LS; and QMA SPHEROSIL® LS from Ciphergen Biosystems of Fremont, Calif. Other suitable are the materials sold under the trade names: UNOSPHERE, MACRO-PREP (including HIGH Q, HIGH S, DEAE, and CM), and AG and Bio-Rex from Bio-Rad Laboratories of Hercules, Calif. Still more suitable commercially available ion exchange materials are sold under the trade names: DEAE-TRISACRYL®, DEAE SEPHAROSE®, DEAE-CELLULOSE, DIETHYLAMINOETHYL SEPHACEL®, DEAE SEPHADEX®, QAE SEPHADEX®, AMBERJET®, AMBERLITE®, CHOLESTYRAMINE RESIN, CM SEPHAROSE®, SP SEPHAROSE®, SP-TRISACRYL®, CELLULOSE PHOSPHATE, CM-CELLULOSE, CM SEPHADEX®, SP SEPHADEX®, and AMBERLITE® from Sigma-Aldrich Co. of St. Louis, Mo. Other commercial sources for ion exchange materials include Amersham Biosciences (www.amersham.com). Still other materials will be familiar to those having skill in the art of protein purification.

Materials suitable for exploiting hydrophobic interactions (hydrophobic interaction chromatography, “MIC”) include those sold under the trade names: PHENYL SEPHAROSE 6 FAST FLOW, BUTYL SEPHAROSE 4 FAST FLOW, OCTYL SEPHAROSE 4 FAST FLOW, PHENYL SEPHAROSE HIGH PERFORMANCE, PHENYL SEPHAROSE CL-4B, OCTYL SEPHAROSE CL-4B, SOURCE™ 15ETH, SOURCE 15ISO, and SOURCEPHE from Amersham Biosciences of Piscataway, N.J. Also available are materials sold as FRACTOGEL® EMD PROPYL (S) AND FRACTOGEL® EMD PHENYL I (S) from VWR International (www.chromatography.uk.co). Still other commercially available HIC materials include the materials sold under the trade names: TOYOPEARL and TSKGEL from Tosoh Bioscience LLC of Montgomeryville, Pa. An equivalent material is sold commercially under the trade name MEP HYPERCEL (Ciphergen Biosystems, Fremont, Calif.). Still other materials will be familiar to those having skill in the art of protein purification.

Affinity materials include any materials effective to attract and sorb biomolecular components on the basis of structural interactions between a biomolecular component and a ligand such as: antibody-antigen, enzyme-ligand, nucleic acid-binding protein, and hormone-receptor. The interactions can be between naturally occurring or synthetic ligand and a biomolecular component. The ligands can be either mono-specific (e.g., a hormone or a substrate) or group-specific (e.g., enzyme cofactors, plant lectins, and Protein A). Examples of common group-specific ligands suitable for the present invention are provided in Table 1.

TABLE 1

| Ligand(s) | Target(s) |
|---|---|
| 5′-AMP, 5′-ATP | Dehydrogenases |
| NAD, NADP | Dehydrogenases |
| Protein A | Immunoglobulins |
| Protein G | Immunoglobulins |
| Lectins | Polysaccharides, Glycoproteins |
| Histones | DNA |
| Heparin | Lipoproteins, DNA, RNA, clotting factors |
| Gelatin | Fibronectin attachment factors |
| Lysine | rRNA, dsDNA, Plasminogen |
| Arginine | Fibronectin attachment factors |
| Benzamidine | Serine proteases |
| Polymyxin | Endotoxins |
| Calmodulin | Kinases |
| Cibacron Blue | Kinases, Phosphatases, Dehydrogenases, Albumins |
| Boronic acid | Biomolecules containing cis-diols (RNA, |
| | glycoproteins) |

Thus, a wide variety of biomolecular materials can be adsorbed using affinity materials. Commercially available affinity materials include those sold under the trade names: PROTEIN A CERAMIC HYPERD® F, BLUE TRISACRYL® M, HEPARIN HYPERD® M, and LYSINE HYPERD® from Ciphergen Biosystems (Fremont, Calif.). Still other commercially available materials are provided by commercial suppliers including Amersham Biosciences (www.amershambioscience.com) and Sigma-Aldrich (www.sigmaaldrich.com). Still other materials will be familiar to those having skill in the art of protein purification.

In some embodiments of the invention, the affinity materials are derived from reactive dyes are used to create sorbents. Dye-ligand sorbents are often useful for binding proteins and enzymes that use nucleic acid cofactors, such as kinases and dehydrogenases; but other proteins, including serum albumins, can be sorted efficiently with these sorbents as well. Examples of suitable commercially available materials include those sold under the trade names REACTIVE BLUE, REACTIVE RED, REACTIVE YELLOW, REACTIVE GREEN, and REACTIVE BROWN (Sigma-Aldrich); DYEMATRIX GEL BLUE, DYEMATRIX GEL RED, DYEMATRIX GEL ORANGE, and DYEMATRIX GEL GREEN (Millipore, Billerica, Mass.); and the Procion dyes known as Blue H-B (Cibacron Blue), Blue MX-R, Red HE-3B, Yellow H-A, Yellow MX-3r, Green H-4G, Green H-E4BD, Brown MX-5BR. Still others will be familiar to those having skill in the art of protein purification.

Useful sorbents can also be constructed from lectins to separate and isolate glycoconjugates, glycans, glycopeptides, polysaccharides, soluble cell components, and cells. Suitable lectins include those shown in Table 2.

TABLE 2

| Lectin | Use(s) |
|---|---|
| Concanavalin A | Separation of glycoproteins, glycoprotein |
| | enzymes, and lipoproteins; isolation of IgM |
| Lens culinaris | Isolation of gonadotropins, mouse H antigens, |
| | detergent-solubilized glycoproteins |
| Tritium vulgaris | Purification of RNA polymerase transcription |
| | cofactors |
| Ricins communis | Fractionation of glycopeptide-binding proteins |
| Jacalin | Purification of C1 inhibitors, separation of |
| | IgA1 and IgA2 |
| Bandeira simplicifolia | Resolution of mixtures of nucleotide sugars |

Immunoaffinity materials can be made using standard methods and materials known to those having skill in the protein purification arts (See, e.g., Protein Purification Protocols). Commercially available immunoaffinity material include those sold by Sigma-Aldrich (www.sigmaaldrich.com) and Amersham Biosciences (www.amersham.com). Similarly, metal-ion affinity (IMAC) materials can be prepared using know materials and methods (See, e.g., Protein Purification Protocols.), or purchased commercially (e.g., from Sigma-Aldrich (www.sigmaaldrich.com) or Amersham Biosciences (www.amersham.com)). Common metal include Ni(II), Zn(II), and Cu(II). Some examples of these materials are shown in Table 3.

TABLE 3

| Chelator Ligand | Metal |
|---|---|
| Iminodiacetate (IDA) | Transition Metals |
| 2-Hydroxy-3-[N-(2- | Transition Metals |
| pyridylmethyl)glycine]propyl | |
| α-Alkyl nitrilotriacetic acid | Transition Metals |
| Carboxymethylated aspartic acid | Ca⁺² |
| Ethylenediamine (TED) | Transition Metals |
| (GHHPH)ₙG* | Transition Metals |

*The letters G and H refer to standard amino acid notation: G = glycine, and H = histidine.

The synthesis of hydroxyapatite (HT/HTP) and thiophilic (TAC) sorbents will also be familiar those having skill in the protein purification arts (See, e.g., Protein Purification Protocols). Commercial sources include Bio-Rad of Hercules, Calif. (trade name CHT), Ciphergen Biosystems of Fremont, Calif. (trade name HA ULTROGEL®), and Berkeley Advanced Biomaterials of San Leandro, Calif. (trade name HAP). Thiophilic sorbents also can be made using methods and materials known in the art or protein purification or purchased commercially under the trade names: MEP HYPERCEL (Ciphergen Biosystems, Fremont, Calif.), THIOPUILIC UNIFLOW and THIOPHILIC SUPERFLOW (Clonetech, Palo Alto, Calif.), THIOSORB (Millipore, Billerica, Mass.), T-GEL (Affiland, Ans-Liege, Belgium), AFFI-T (Ken-en-Tec, Copenhagen, Denmark), HI-TRAP (Amersham Biosciences, Piscataway, N.J.), and FRACTOGEL (Merck KgA, Poole Dorset UK).

The above-described sorbent materials have specificities for different biomolecular components. In this regard, the term “specificity” relates to the number of different biomolecular species in a given sample which a sorbent can bind. In one aspect, sorbents can be grouped by their relative degrees of specificity, for example high specificity sorbents, moderate specificity sorbents, and low specificity sorbents. High specificity sorbents include those materials that generally have a strong preference to sorb certain biomolecules or subsets of biomolecules. Often such materials include highly biospecific sorption interactions, such as antibody-epitope recognition, receptor-ligand, or enzyme-receptor interactions. Examples of these sorbents include Protein A-, Protein G-, antibody-, receptor- and aptamer-bound sorbents. Moderate specificity sorbents include materials that also have a degree of bispecific sorption interactions but to a lesser degree than high specificity materials, and include: MEP, MBI, hydrophobic sorbents, and heparin-, dye-, and metal chelator-bound materials. Many “mixed-mode” materials have moderate specificity. Some of these bind molecules through, for example, hydrophobic and ionic interactions. Low specificity sorbents include materials that sorb bimolecular components using bulk molecular properties (such as acid-base, dipole moment, molecular size, or surface electrostatic potential) and include: zirconia, silica, phenylpropylamine cellulose, ceramics, titania, alumina, and ion exchangers (cation or anion).

The progression from high specificity to low specificity serves a particularly useful purpose. In particular, it allows fractionation of the proteins in the sample into largely exclusive groups, but of decreased complexity. As such, the proteins in the various fractions are more easily resolved by the detection method chosen. For example, a low- or moderate-specificity resin might have affinity for or bind to many biomolecules in a sample, including ones in very high concentration. However, by exposing the sample to a high specificity sorbent that is directed to the protein in high concentration before exposing to the moderate-specificity sorbent, one can remove most or all of the high concentration protein. In this way, the set of biomolecules captured by the moderate specificity sorbent will largely or entirely exclude the high concentration biomolecule. This results in a less complex set of proteins captured by the moderate specificity sorbent. The strategy, thus, is to remove at earlier stages biomolecules, e.g., proteins, that would otherwise be captured by sorbents at later stages of the fractionation process so that at each stage, the complexity of the biomolecules passing to the next stage is decreased.

In one embodiment of the invention, the solution of biomolecular components is contacted with at least three different sorbents from among high-, moderate-, or low-specificity sorbents. In some embodiments, the solution will be contacted with one, two, or three or more materials of the same degree of specificity (e.g., two materials of moderate specificity or three materials of low specificity). In another embodiment, the solution is contacted with a plurality of sorbents that define a progression from high specificity to low specificity. In another embodiment, the solution is contacted with a plurality of sorbents that define a progression from high specificity to low specificity. In yet another embodiment, the sorbent materials are arranged to provide a substantially linear progression of specificities. In still another embodiment, the sorbent materials form a substantially contiguous body. In still another embodiment, the sorbents are mutually orthogonal, i.e., the ability of each sorbent is substantially selective for a unique biomolecular component or subset of biomolecular components. In another example, the sorbents are chosen such that at least one sorbent is a high specificity sorbent and at least one other sorbent is either a moderate- or low specificity sorbent. In another embodiment, the sorbents are chosen such that at least one sorbent each is a high specificity sorbent, a moderate specificity sorbent, and low specificity sorbent. In still another embodiment, at least two sorbents are chosen from two classes of high specificity sorbents, moderate specificity sorbents, and low specificity sorbents. In another embodiment, at least two sorbents are high specificity sorbents and at least one sorbent is a low specificity sorbent.

Alternatively, a series of sorbents having the same degree of specificity can be used. In this embodiment, while the sorbents possess the same relative degree of specificity, they have different absolute specificities, i.e. each sorbent individually binds to different numbers of species of bimolecular components in a sample. Thus, when sorbents having the same degree of specificity are utilized, they are arranged to provide a substantially linear progression of adsorption from highest specificity to lowest specificity. A second sorbent has decreased specificity compared with a first sorbent if, when exposed to the same sample, the second sorbent binds more species from the sample than the first sorbent.

For example, in one embodiment each of the sorbents in the series can be a hydophobic sorbent. In this regard, each sorbent comprises a hydrocarbon chain and, optionally, an amine ligand, and the hydrocarbon chain of each sorbent in the series comprises more carbons than the previous sorbent. Suitable terminal binding functionalities include, but are not limited to, primary amines, tertiary amines, quaternary ammonium salts, or hydrophobic groups. The sorbents can comprise, for example, hydrocarbon chains selected from the group consisting of C1, C2, C3, C4, C5, C6 and so on.

Among other properties, proteins are characterized by their hydrophobic degree (called also hydrophobic index) which is the result of the content and the sequence of lipophilic amino acids such as leucine, isoleucine, valine and phenylalanine. As a function of the hydrophobic degree, proteins associate with hydrophobic interaction adsorbents in the presence of lyotropic salts. The strength of adsorption depends on both the hydrophobic character of the sorbent and the concentration of lyotropic salts. When sorbents are designed in such a way so that they are capable to associate proteins in physiological conditions, the only variable will be the structure of the sorbent itself. The hydrophobicity degree of a sorbent depends on the length of the hydrocarbon chain of the ligand used and its density. However, if the ligand density is fixed only the length of the hydrocarbon chain would play the role of adsorbent moiety. In practice it is possible to synthesize sorbents with ligands of different chain length and the same ligand density. If the ligand is selected among those that produce adsorption in physiological conditions, it is possible to put in place a system where the discrimination will be dependent only on the solid phase.

If a slightly hydrophobic sorbent is loaded with a group of proteins, only the most hydrophobic will be captured and all others will be found in the flowthrough. Then if the supernatant will be contacted with a sorbent of medium hydrophobicity, proteins of medium hydrophobicity will be captured and others will be found in the supernatant. Finally if this second supernatant containing the least hydrophobic proteins is contacted with a very hydrophobic sorbent all other proteins will be adsorbed.

In this situation it is possible to superimpose various hydrophobic sorbents and load proteins throughout the different layers. The sequence of superimposed sorbent should be composed of the mildest hydrophobic sorbents first, followed by a sequence of sorbents of growing hydrophobicity degree. To have the system work as expected, it is necessary to work in under-loading conditions so that the first layer of the column will deplete for the most hydrophobic species, the second layer will then remove a group of less hydrophobic species and so on. The last section of sorbent (the most hydrophobic) will finally remove the least hydrophobic proteins.

Adsorption is operated using the same buffer for all column sections; the preferred buffer is a physiological phosphate buffer containing 0.15 M sodium chloride. To this buffer modifiers could be added to modulate the conditions for protein adsorption (see variations to the general method).

The sorbent is made using hydrocarbon chains of different length so that to drive the degree of hydrophobicity of the columns sections. More particularly the hydrophobic ligands are primary amines on one extreme and a hydrophobic moiety at the other extremity. The first ligand of the series is methylamine, followed by ethylamine, propylamine, butylamine, pentylamine, hyxylamine and so on. The longest hydrophobic amine of practical interest in the present application is octadecylamine.

Amine groups at the extremity of the ligand induces protein adsorption without addition of lyotropic acid. This so-called physiological hydrophobic interaction adsorbent (HIC) is described in international patent application No. PCT/US2005/001304. However, other linkers can easily be used such as thio-ethers (“S” bridges) and ethers (“O” bridges).

Preferred matrix material for the preparation of the solid sorbents is cellulose and other polysaccharides. The preferred activation method for the introduction of the hydrophobic ligand is allyl bromide.

A typical example of separation of proteins by their hydrophobicity degree is as follows:
Prepare aliphatic hydrophobic supports with the following hydrocarbon chains: C2, C4, C8.
Pack each sorbent is three superimposed Promega columns each filled with 125 μL of sorbent.
The columns are then equilibrated with a physiological phosphate buffered saline followed by the injection of 200 μliters of albumin-depleted serum (protein concentration: 5 mg/mL). The sample is then pushed through the sectional columns using PBS. Once the adsorption phase is over, sectional columns are disconnected and proteins adsorbed on each of them are eluted using a mixture of TFA/ACN/Water (0.8%-20%-79.2%). Collected proteins are then analyzed by SELDI MS.

Types of hydrophobic ligands useful in this method include aliphatic linear chains such as methyl through octadecyl; they can be branched aliphatic hydrocarbon chains; they can be cyclic structures or aromatic hydrophobic structures. They can also be combinations of aliphatic and aromatic structures.

Preferred embodiments of the invention conform to the general formula (I): 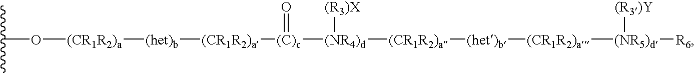
as described generally above. In this formula, R₁, R₂, R₄, and R₅ are independently selected from H, C₁₋₆-alkyl, C₁₋₆-alkoxy, C₁₋₆-alkyl-C₁₋₆-alkoxy, aryl, C₁₋₆-alkaryl, —NR′C(O)R″, —C(O)NR′R″, and hydroxy. Preferably, R₁, R₂, R₄, and R₅ are independently selected from H and C₁₋₆-allyl. The most preferred embodiments are those in which R₁ and R₂ are H, while R₄ and R₅ are C₁₋₆-alkyl.

Depending upon the desired terminal binding functionality, R₆ is selected from the group consisting of H, C₁₋₄-alkyl, aryl, C₁₋₆-alkaryl, —C(O)OH, —S(O)₂OH, and —P(O)(OH)₂. The terminal binding functionality as a whole is thus represented generally by —(NR₅)(R_{3′})Y—R₆ in formula (I). In one preferred embodiment, for example, d′ is 1, thus giving the terminal binding functionality as an amine (when (R_{3′})Y is absent) or a quaternary ammonium salt (when (R_{3′})Y is present). In these embodiments, R₆ is preferably C₁₋₆-alkyl.

In other embodiments, d′ is 0, thus providing for a terminal binding functionality that is represented predominantly by R₆. In these cases, R₆ is preferably chosen from H, C₁₋₆-alkyl, aryl, and C₁₋₆-alkaryl groups when a hydrophobic terminal binding functionality is desired. Where the terminal binding functionality is a cation exchange group, R₆ is accordingly chosen from —C(O)OH, —S(O)₂OH, and —P(O)(OH)₂.

The moieties (R₃)X and (R_{3′})Y, when they are present in formula (I), form quaternary ammonium salts with the respective nitrogen atoms to which each moiety is bound. As required by formula (I), X and Y represent anions. No particular requirements restrict the identity of these anions, so long as they are compatible with the prescribed use of the chromatographic material. Exemplary anions in this regard include fluoride, chloride, bromide, iodide, acetate, nitrate, hydroxide, sulfate, carbonate, borate, and formate.

The balance of formula (I), therefore, generally represents the hydrophobic linker. Consistent with the definition of a hydrophobic group as defined hereinabove, the linker is hydrophobic overall, which property is achieved preferably by incorporating alkylene chains into the linker, corresponding to the selection of a, a′, a″, and a′″. Preferably, at least one of a, a′, a″, and a′″ is 2 or 3, more preferably at least two of a, a′, a″, and a′″ are 2 or 3, and most preferably a is 3 while a′ is 2, 3, 4, 5, or 6.

In preferred embodiments, the linker is thiophilic in addition to being hydrophobic. Accordingly, one or both of het and het′ in formula (I) are chosen from increasingly thiophilic groups —S—, —S(O)—, and —S(O)₂—, S being most preferred. In the most preferred chromatographic material, het is S while het′ is absent.

The inventors have discovered that certain subsets of chromatographic materials are particularly efficacious. This is so because the materials present significant patches or regions of hydrophobicity in the hydrophobic linker, which property is generally achieved by coupling alkylene fragments together. Thus, at least two of (CR₁R₂)ₐ, (CR₁R₂)_{a′}, (CR₁R₂)_{a″} and (CR₁R₂)_{a′″} represent two unsubstituted ethylene groups (i.e., —CH₂—CH₂—). Alternatively, the hydrophobic linker can comprise at least two unsubstituted propylene groups. That is, at least two of (CR₁R₂)ₐ, (CR₁R₂)_{a′}, (CR₁R₂)_{a″} and (CR₁R₂)_{a′″} represent two propylene groups (i.e., —CH₂—CH₂-CH₂—). In another embodiment, the hydrophobic linker can comprise at least one unsubstituted ethylene group and at least one mono-substituted propylene group. For example, at least one of (CR₁R₂)ₐ, (CR₁R₂)_{a′}, (CR₁R₂)_{a″} and (CR₁R₂)_{a′″ is —CH}₂—CH₂— and at least one is —C₃H₅(OH)—. In another embodiment, the hydrophobic linker can comprise at least two mono-substituted propylene groups. For example, at least two of (CR₁R₂)ₐ, (CR₁R₂)_{a′}, (CR₁R₂)_{a″} and (CR₁R₂)_{a′″} are —C₃H₅(OH). In these embodiments the alkylene groups can be separated by a heteroatom or a group comprising a heteroatom, such as —O—, —S—, —NH— or —C(O)N(H)—. All combinations of these are contemplated.

More specifically, one embodiment incorporates an unsubstituted propylene group and an unsubstituted ethylene group that are separated by het or het′ in general formula (I), in which, for example, a (or a″) is 3, a′ (or a′″ is 2), and b (or b′) is 1. In this embodiment, it is possible, however, to substitute the propylene group with one hydroxyl group and maintain the overall hydrophobicity of the linker.

In another preferred embodiment, the hydrophobic linker comprises two unsubstituted propylene groups that are separated by het or het′. Thus referring to general formula (I), a and a′ are both 3 while b is 1, or a″ and a′″ are both 3 while b′ is 1.

In yet another preferred embodiment, the hydrophobic linker comprises an unsubstituted propylene group and at least an unsubstituted pentylene group that are separated by het, thus corresponding to a being 3, a′ being 5, and b being 1 in general formula (I). In this embodiment, the propylene group can be substituted once with a hydroxyl group.

In still another preferred embodiment, the hydrophobic linker comprises two unsubstituted propylene groups that are separated by one amino moiety. Referring therefore to general formula (I), a or a′ is 3, the other being 0; a″ or a′″ is 3; het and het′ are absent; and c is 0 while d is 1.

In general formula (I), the wavy line represents the solid support to which the hydrophobic linker is attached. It is understood for the purpose of clarity, however, that general formula (I) depicts only one (1) linker-terminal binding functionality as being tethered to the solid support. The inventive chromatographic materials actually exhibit linker-terminal binding functionality densities of about 50 to about 150 μmol/mL chromatographic material, preferably about 80 to about 150 μmol/mL, and more preferably 100 to about 150 μmol/mL.

The type of linker that attaches the ligand to the matrix, which makes it possible to function at physiological ionic strength include a nitrogen, a sulfur group or an oxygen atom.

The activation of the solid matrix can be accomplished using the well known chemical approaches used in affinity chromatography. The preferred one involves the use of allyl groups. This is obtained by reacting the solid phase matrix with allyl-bromide or allyl-glycydyl-ether.

Buffers for protein loading is most generally a physiological buffer such as PBS. A large number of variations are possible in terms of pH, ionic strength and nature of components. Modifiers to the adsorption buffer is also a possibility especially when the modulation of the hydrophobic association is necessary (weaken the hydrophobic association). This can be accomplished by adding to the initial buffer detergents, alcohols, urea, thiourea, guanidine, etc.

Desorbing solutions are composed of any possible chemical component capable to elute proteins from the sorbent. Most generally this is composed of a hydro organic mixture of acidic pH such as trifluoroacetic acid, acetonitrile and water. Desorption solutions may however be of alkaline pH and containing alcohols or detergents or chaotropic agents.

Superimposed layers can go from two layers up to ten or even 20 layers of different hydrophobic sorbents of growing hydrophobic degree.

Devices used to apply the described principle can be superimposed columns where the outlet of the upper column is directly linked to the inlet of the following column. It can be a set of superimposed 96-well filtration plate or any possible device that allows injecting sequentially a protein solution throughout a series of solid phase sorbents in packed and slurry mode.

Proteins to separate by using the described method are from biological fluids such as serum, urine, CSF; it can be a tissue soluble extract. A specific aspect contemplated by this principle is the separation of components from membrane extracts. They can be done in the presence or urea and then loaded on the sequence of the columns.

The above-described materials are used in any manner and with any apparatus known to those of skill in the art to separate biomolecular materials from complex mixtures of such. Commonly known formats for using these materials include: column chromatography, medium-pressure liquid chromatography, high-pressure liquid chromatography, flat surfaces or other two-dimensional arrays (such as PROTEINCHIP® arrays from Ciphergen Biosystems of Fremont, Calif.), or 96-well filtration plates. The latter are useful for parallel fractionations. The apparatus used for separation may further include the addition of an electric potential to allow isoelectric focusing. Still more formats will be know to those of skill in the protein purification arts.

In one embodiment, the sorbents are chosen such that the biomolecular materials of the greatest concentrations are removed first. For example, the protein composition of human serum includes upwards of 90% of the following: albumin, IgG, transferrins, α-1 anti-trypsin, IgA, IgM, fibrinogen, α-2-macroglobulin, and complement C3. About 99% of human serum further includes: apolipoproteins A1 and B; lipoprotein A; AGP, factor H; ceruloplasm; pre-alburnin; complement factor B; complement factors C4, C8, C9, and C19; and α-glycoprotein). The reaming 1% comprise the so-called deep proteome. Arranging the sorbents such that a Protein A sorbent and a Cibacron Blue sorbent are the first two sorbents can reduce the dynamic range of human serum from approximately 10⁸ to about 10⁵, thereby allowing capture of lower abundance biomolecular components for identification and quantitation. Often, placing a sorbent such as phenylpropylamine cellulose at the end of the column is useful to catch any remaining biomolecular components in the sample. Generally, if the initial sorbent(s) are too general (i.e., have low specificity), then too much material can be sequestered with the first two sorbents, which degrades the usefulness of the remaining sorbents. However, if the initial sorbents are too specific (i.e., have high specificity), then the efficiency of the remaining sorbent materials can be reduced by a large sample dynamic range. In one embodiment, the sorbents are chosen such that the first sorbent, or first and second sorbents combined, provide a reduction in the dynamic range of the sample by a factor of at least 10, more specifically a factor of at least 100, and, still more specifically a factor of at least 1,000.

Thus, the invention provides a method for depleting highly abundant biomolecular components from a complex mixture that includes a plurality of such biomolecular components of different concentrations, comprising: contacting said complex mixture with a biospecific adsorbent material to provide thereby a low-abundance complex mixture; and contacting said low-abundance complex mixture with, in sequence, a mixed-mode adsorbent material and a non-specific adsorbent material to provide thereby a depleted complex mixture that comprises those biomolecular components having concentrations of less than about 5% of the concentrations of said highly abundant biomolecular components. In another embodiment, the method of the invention provides a complex mixture that comprises those biomolecular components having concentrations of less than about 1% of the concentrations of said highly abundant biomolecular components. In still another embodiment, the method of the invention provides a depleted complex mixture that comprises those biomolecular components having concentrations of less than about 0.1% of the concentrations of said highly abundant biomolecular components. In yet another embodiment, the method of the invention provides a depleted complex mixture that comprises those biomolecular components having concentrations of less than about 0.01% of the concentrations of said highly abundant biomolecular components. In still yet another embodiment, the method of the invention provides a depleted complex mixture that comprises those biomolecular components having concentrations of less than about 0.001% of the concentrations of said highly abundant biomolecular components.

In still another embodiment the invention provides a complex mixture as described herein, in which the depleted mixture is enriched for species which, in the original mixture, comprised less than 5% of the total protein mass; more specifically, less than about 1% of the total protein mass; still more specifically less than about 0.1% of the total protein mass; yet more specifically less than about 0.01% of the total protein mass; and still yet more specifically less than about 0.001% of the total protein mass.

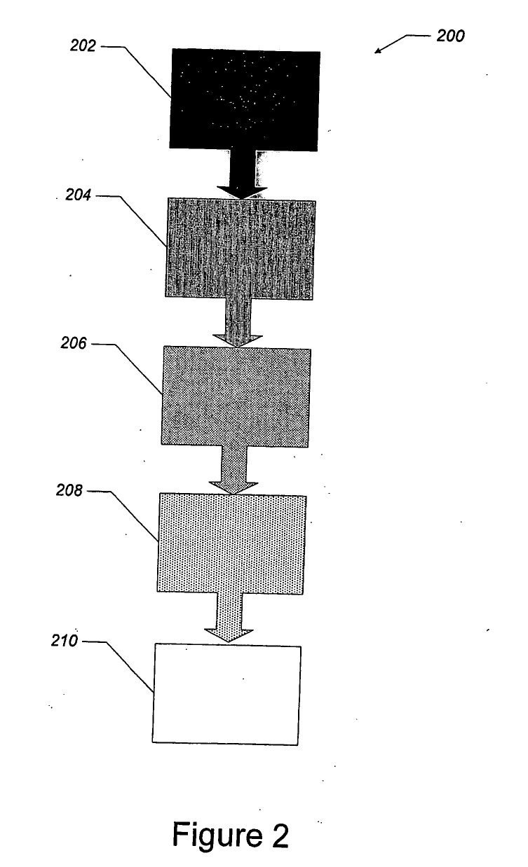

This aspect of the invention is illustrated in FIG. 2 at 200, in which a complex sample, e.g., human serum, having at least one biomolecular component of large concentration, such as immunoglobulins (IgG, transferrin, α-1 anti-trypsin, IgA, IgM, and haptoglobin) and albumin, is sorbed by a first sorbent 202 which reduces the dynamic range of component concentrations. For example, sorbent 202 can be Protein A, which has a high specificity for immunoglobulins. Exposure of this material to a second sorbent 204 provides further reduction of dynamic range. Such a sorbent can be another having a large ability to sorb additional immunoglobulins, albumin, and clotting factors, or other species of predominance. One example of such a sorbent is Cibachron Blue or heparin. Such sorbents can reduce dynamic range by factors of 10, or 100, or 1,000 as discussed above. Further exposure to sorbent 206 allows capture of the lesser abundant components. Such sorbents can include mixed-mode materials, such as dyes, chelators, or antibodies directed to specific components. The remaining components in the sample are exposed to a low specificity material 208, such as phenylpropylamine, silica, or zirconia. Finally, the remaining eluent is collected at 210.

For example, serum is a complex biological fluid having a large dynamic range of protein concentrations (˜10⁸). Proteins at the highest concentrations include albumins and immunoglobulins. Accordingly, as illustrated in the Examples, a useful sequence of sorbents places those sorbents having a large ability to remove the dominating proteins in the early stages of the fractionation (e.g., at the top of the column) to remove those proteins from the sample first. Following the first sorbent(s) are moderate- and low specificity sorbents that are effective to remove the lower abundance proteins. However, high specificity sorbents, such as resin-mounted antibodies can be used to trap specific lower abundance biomolecules as well. One sequence described in greater detail below is: Protein A-HyperD (captures immunoglobulins)—Blue Trisacryl M (captures albuminy Heparin-HyperD—MEP-HyperCel—Green 5-agarose—Zirconia oxide—Phenylpropylamine-Cel. Protein A removes immunoglobulins. Blue Tris Acryl M removes albumin. Heparin-HyperD removes various clotting factors (from plasma). MEP-HyperCel removes proteases. Green 5 (a mixed-mode sorbent) removes proteins having net positive surface charges. Of course, other complex biological fluids also can be prefractionated using the disclosed methods.

Once the sorbents have been chosen and packed into a column, or otherwise configured for use, a buffer solution is prepared for the sample solution. In general, the buffer can be any buffer solution that is compatible with the various sorbent materials used in the fractionation, i.e., such that the buffer does not substantially degrade the ability or performance of the sorbent. Such considerations will be familiar to those of skill in the protein purification arts. In one embodiment of the invention, the buffer has neutral pH or a pH value within physiological limits. The latter is useful for samples derived from bodily fluids, such as blood. In a more particular embodiment, the buffer has a pH=8 and includes 0.1 M Tris-HCl, 16% PBS (phosphate-buffered saline), and water. In another embodiment, the buffer is determined by first estimating a buffer formulation using the technical characteristics of the sorbents, and then iteratively adjusting the buffer to optimize the fractionation of a sample run on the column. Such optimization includes determining the number of spots produced on a subsequent 2D-gel or the number of peaks identified by a mass spectrographic analysis such as Surface Enhanced Laser Desorption Ionization (SELDI). The test material or sample may also be spiked with a known material to determine if that material is substantially sorbed by a particular sorbent material. The buffer can be adjusted to a final formulation using such isolation as a formulation criterion. Other criteria can be used, as will be apparent to those of skill in the protein purification arts. For example, if the sample is from blood, one criterion may be the efficiency of albumin or immunoglobulin removal from the sample by the first sorbent material.

Following determination of the buffer, the sample solution is prepared and the column loaded with the solution. Generally, the determination of the sample concentration and amount of solution loaded on the column will be determined using techniques known to those of skill in the protein purification arts. In some cases, the operator will prepare one, two, or more test columns to determine an optimal concentration and loading. In one embodiment, the sample is diluted about five-fold to provide about a total volume of 100 μL and loaded onto prepared 96-well plates. In another embodiment, about 20 μL of a sample is diluted to about 200 μl and pumped onto a prepared column using a syringe pump.

After loading, the solution is allowed to traverse the sorbents in the column or stacked plates (or other appropriate apparatus) such that biomolecular components in the sample contact and either captured or sequestered by a sorbent or pass to the next sorbent. In one embodiment, each subset of biomolecular materials is isolated with substantially a single sorbent such that no substantial quantity of biomolecular components elutes from the apparatus.

In one embodiment, the sorbents form a contiguous biomolecular-sequestering body. Thus, the contacting of a complex mixture to a series of sorbents occurs as a continuous process, without interruption or additional processing between the different sorbents in the series. Following capture of each subset of biomolecular components, each sorbent material can be excised from the body (e.g., by cutting) for subsequent processing of the biomolecular components sorbed thereby. Alternatively, using a segmented column, such as that sold under the trade name WIZARD, individual elements holding the sorbent and sorbed materials can be removed for later processing. Thus, each sorbent-containing segment in the column is detachable.

Accordingly, in one aspect, there is provided an apparatus comprising at least three detachable segments wherein each segment comprises a sorbent having a different adsorption specificity and wherein the segments are arranged in a progression of decreasing specificity of the sorbents. In one embodiment, the segments are physically attached to each other. In another, the segments are connected by an intermediary, such as a tube or conduit to form a fluid path. In this embodiment, each segment ideally comprises attachment means for in-flow and out-flow tubes and means for retaining the sorbent in the segment. A multi-well filtration plate can be used in this manner. In this regard, the fluidics device disclosed in U.S. Provisional Application No. 60/684,177, filed on May 25, 2005, which is hereby incorporated by reference, provides a multi-well plate with detachable segments and would be useful as a platform in the present invention.

Following isolation of a sorbent, the sequestered biomolecular material can be eluted using known materials and techniques that are appropriate for the sorbent and biomolecular material. Examples of suitable elution methods include, but are not limited to: exposure to water, a chaotropic agent, a lyotropic agent, an organic solvent, change in ionic strength, change in pH, change in temperature, change in pressure, or a combination of any two or more of the foregoing.

Following elution, the isolated biomolecular materials can be subjected to further operations. In one embodiment, the eluted biomolecular components are subjected to a second separation procedure. The second separation procedure can be another fractionation as provided by the present invention, a conventional fractionation procedure, one-, two-, or multi-dimensional gel electrophoresis, mass spectrometry, and medium- or high-pressure liquid chromatography. In another embodiment, the chemical identity of a biomolecular component is determined. Such determination can be done by fluorometry, mass spectrometry (including deposition of the component material on a SELDI probe followed by laser desorption-ionization mass spectrometry), one-, two-, or multi-dimensional gel electrophoresis, and medium- or high-pressure liquid chromatography. Other suitable methods include amino- or nucleic acid sequence analysis, nuclear magnetic resonance, and X-ray crystallography individually or in combination. Still more will be apparent to those of skill in the protein chemistry arts.

In Example 1, 75 μL of the sorbents Protein A, zirconia, Heparin, MEP, GREEN 5, and 150 μL of the sorbents Blue Trisacryl and phenylpropylamine cellulose, were packed into the individual elements of a WIZARD mini-column. The sorbents were equilibrated with 200 μL per well of the binding buffer (PBS (16v)/1 M Tris.HCl (pH8, 9v)/H₂O (75v)). A sample volume of 100 μL (five-fold dilution) of a solution of biomolecular components was passed through the column. The column elements were isolated and the sorbed materials were eluted. The eluates were analyzed by mass spectrometry and the results were compared to the same mass spectrographic analysis of a sample derived using a single column. The method of the invention provided almost two-fold more peaks (89% more) than the prior art method.

The present invention also provides apparatuses and kits for fractionating complex mixtures of biomolecular components in accordance with the description provided above.

In one aspect, the present invention provides an apparatus for prefractionating a complex mixture of biomolecular components. In one embodiment, the apparatus includes a plurality of sorbents described above having different adsorption specificities for different biomolecular components. The sorbents are coupled serially and in fluidic communication such that introduction and passage of the mixture in a buffered solution as described above is effective to remove at least a portion of the components from the complex mixture. Various embodiments of these elements can be provided as described above. For example, the sorbents can be arranged to provide a progression of specificities for a type of biomolecular component. Such a progression can be linear. The sorbents can also be provided as a substantially contiguous component-sequestering body. The sorbents can be arranged in a columnar assemblage or in an array of columns, such as provided by a series of 96-well plates. In another embodiment, the sorbents are chosen for the apparatus to include: (a) a high specificity sorbent, (b) a moderate specificity sorbent material, and (c) a low specificity sorbent material.

In another aspect, the invention provides a kit comprising a plurality of sorbents characterized by different adsorption specificities for different biomolecular component types and a compatible buffer. The combination is chosen such that when the materials are coupled in a series arrangement, introduction and serial passage of a buffered solution including (i) said complex mixture and (ii) said buffer through said series arrangement of materials is effective to capture substantially all of said plurality of biomolecular components from said complex mixture. In another embodiment, the sorbents are chosen for the apparatus to include: (a) a high specificity sorbent, (b) a moderately specific sorbent material, and (c) a low specificity sorbent material.

### EXAMPLES

The following examples are provided to illustrate certain embodiments of the present invention as a guide to understanding the invention and are in no way to be interpreted as limiting the scope of the invention. Descriptions of the reagents and general procedures are provided below.

Materials

The vacuum unit came from Whatman (Clifton, N.J., USA). The MICROMIX mixer was from DPC (Los Angeles, Calif., USA). The MINIPULS III peristaltic pump was from Gilson (Middleton, Wis., USA). Q-HYPERD F®, PROTEIN A CERAMIC HYPERD®, BLUE TRISACRYL®, HEPARIN HYPERD®, MEP-HYPERCEL®, immobilized Green 5 on cellulose, zirconia and phenylpropylamine cellulose sorbents were purchased from commercial sources (Ciphergen/BioSepra, 48 Avenue des Genottes, Cergy St. Christophe, France). SILENT SCREEN LOPRODYNF filter plates were purchased from NUNC (Rochester, N.Y., USA). WIZARD mini-columns were purchased from Promega (Madison, Wis., USA). Sinapinic acid (SPA) was purchased from Ciphergen Bioinstruments (Fremont, Calif., USA). One molar Tris-HCl pH 8 stock buffer was purchased from Invitrogen (Carlsbad, Calif., USA). Human serum was purchased from Intergen (Norcross, Ga., USA). Bovine insulin, PBS buffer, Trifluoro-acetic acid (TFA), isopropanol (IPA), acetonitrile (ACN), ammonia 29% (NH₄OH) solution were purchased from Sigma-Ultra. Urea, CHAPS, Trisma base, octyl-glucopyranoside (OGP), HEPES, sodium acetate, and sodium citrate were purchased from Sigma-Aldrich (St. Louis, Mo., USA).

Preparation of Denatured Human Serum Samples

A sample of denatured human serum was prepared by combining 2 ml of human serum with 2.5 ml of a 9 M urea-2% CHAPS solution over a period of about one hour at room temperature. The solution was aliquoted and frozen. Then 0.4 ml this denatured serum was added of 36 μl of a 1M Tris-HCl pH 9 stock buffer, 100 μl of the 9 M urea-2% CHAPS solution, and 364 μl of DI water to achieve a total 20% dilution of the human serum.

Spiking of Bovine Insulin in Human Serum

A 1 μM solution of bovine insulin (Sigma) in 0.1M Tris-HCl (pH8) was added to native- or denatured human serum in to obtain a final insulin concentration of 100, 10, or 1 femtomoles per microliter (fMol/μL) of serum.

SELDI-MS Analysis

A sample pool of the solutions having a volume of 30 μl was half-diluted in a binding (0.5M NaCl in 0.1M sodium phosphate pH 7 ([MAC30), 0.1M Sodium acetate pH 4 (CM10), 50 mM Tris-HCl pH 9 (Q10), and 0.1% TFA, 10% acetonitrile (H50)) corresponding to the ProteinChip array that was used (IMAC30, CM10, Q10 or H50 arrays). After 30 min. incubation at RT, the array was washed twice with 150 μL of the binding buffer and extensively washed with deionized (DI) water. A 0.5 μL aliquot of Sinapinic (SPA) saturated solution was added two times before reading on the ProteinChip reader. Counting of unique peaks was performed on each Protein Chip array using ProteinChip Software 3.2.0 (available from Ciphergen Biosystems, Fremont, Calif.). Peak counting after clustering of the four arrays consisted to count only once the peaks of same mass that were detected on more than one array. IMAC30, CM10, Q10 and H50 ProteinChip arrays were functionalized by nitrolo-acetic-, carboxymethyl-, quaternary ammonium-, and C 16-hydrophobic moieties, respectively.

Description of the Fractionation Protocols

### Example 1

### Multiple Chemistry Fractionation of Human Serum on a 96-Well Filter Plate

Each filter-plate was dedicated to only one sorbent chemistry and filled with 75 μL of the same sorbent per well, except for Blue-Trisacryl and phenylpropylamine cellulose where 150 μl of each were used per well. Each sorbent was equilibrated by adding 200 μL per well of the binding buffer (PBS (16v)/1 M Tris.HCl (pH8, 9v)/H₂O (75v)), with 5 min. soaking followed by vacuum removal of the buffer. The equilibration procedure was repeated four times to achieve a complete equilibration. The sorbents were allocated to the plates as showing in Table 4.

TABLE 4

| Plate Number | Sorbent |
|---|---|
| 1 | Protein A |
| 2 | Blue Trisacryl |
| 3 | Blue Trisacryl |
| 4 | Heparin |
| 5 | Mep |
| 6 | Green 5 |
| 7 | Zirconia |
| 8 | Phenylpropylamine cellulose |

An aliquot of 100 μL of human serum (bovine insulin-spiked or unadulterated) that had been diluted five-fold in 0.1 M Tris-HCl pH8 buffer was added to the wells of plate 1 that had been filled with Protein A sorbent and incubated for 20 min. on the mixer (intensity set to level 7). The sorbent supernatant was then filtered-off directly on the plate 2 (Blue Trisacryl) placed on the vacuum unit as the receiving plate. Plate 1 received 160 μl of the binding buffer to perform a first wash. Plates 1 and 2 were each incubated on the mixer for 20 min. The supernatant of plate 2 was transferred to plate 3 (Blue Trisacryl) as described above, and the supernatant of plate 1 was transferred to plate 2. Then plate 1 received a second aliquot (160 μL) of the binding buffer for a second wash The three plates 1-3 were incubated on the mixer for 20 min. The same procedure was continued where the supernatants from any plate “N” was vacuum-transferred to the plate “N+1”. Plate 1 after vacuum-transfer of its supernatant was washed a total of five times with the binding buffer.

All the supernatants from the final plate 8 (phenylpropylamine) were transferred to a clean 96-plate to give the flow-through fractions ready for analysis. The elution of bound material was performed by addition of 160 μl of either a solution of TFA (0.4v)/H₂O (39.6v)/ACN (3.3v)/IPA (6.7v) for plates 1, 5, and 8, or a solution of NH₄OH (4v)/H₂O (36v)/ACN (3.3v)/IPA (6.7v) for plates 2, 3, 4, 6, and 7, followed by incubation of all the plates on the mixer for 20 min. After the vacuum-transfer of all the eluates in clean and labeled 96-well plates to give the elution fractions, the same elution operation was repeated a second time. All the eluates (2×160 μL) coming from a same well were pooled, frozen, and lyophilized in the plate. All the lyophilized fractions were dissolved in 100 μL of 25 mM Tris-HCl (pH7.5) before analysis.

Reference Anion Exchange Fractionation Plate of Human Serum (Spiked with Bovine Insulin or Not) On 96-Well Filter Plates

One filter plate was filled with 90 μL of Q-HYPER D F™ per well. Sorbent in each well was equilibrated by addition 200 μL per well of the binding buffer (1 M urea/0.22% CHAPS/50 mM Tris-HCl pH 9) and allowed to soak for 5 min. The buffer was then removed by vacuum. This was repeated four times to achieve a complete equilibration.

A sample volume of 100 μL of denatured human serum (bovine insulin-spiked or straight) diluted five-fold in 40 mM Tris-HCl pH 9 buffer (See described protocol in Section 4.2.5.1) was added to the sorbent incubated for 45 min. on the mixer (intensity setting 7). The sorbent supernatant was then filtered-off directly to a clean 96-well plate to give the flow-through fractions. Then, 100 μL of a 50 mM Tris-HCl pH 9/0.1% OGP buffer was added to the beads and the combination was incubated for 10 min. on the mixer (intensity setting 7). The supernatant was then filtered-off and pooled with the previous flow-through fraction. Then step-elutions by pH decrease were started by the addition of 100 μL of a 50 mM HEPES pH 7/0.1% OGP buffer to the beads with incubation for 10 min on the mixer (intensity setting 7). After vacuum-transfer of the HEPES supernatant in another clean 96-well plate, the same step was repeated; and the two HEPES eluents were pooled together to give 200 μl fractions at pH 7. The same steps (2×100 μl) were repeated for each of the following acidic eluents with 100 mM sodium acetate pH 5, 100 mM sodium acetate pH 4, 50 mM sodium citrate pH 3 and 0.1% TFA/16.6% ACN/33.3% IPA (organic) solutions. At the end of the elution, the six fractions (flow-through, pH 7, pH 5, pH 4, pH 3 and organic) were ready for analysis.

Peak Counting Results

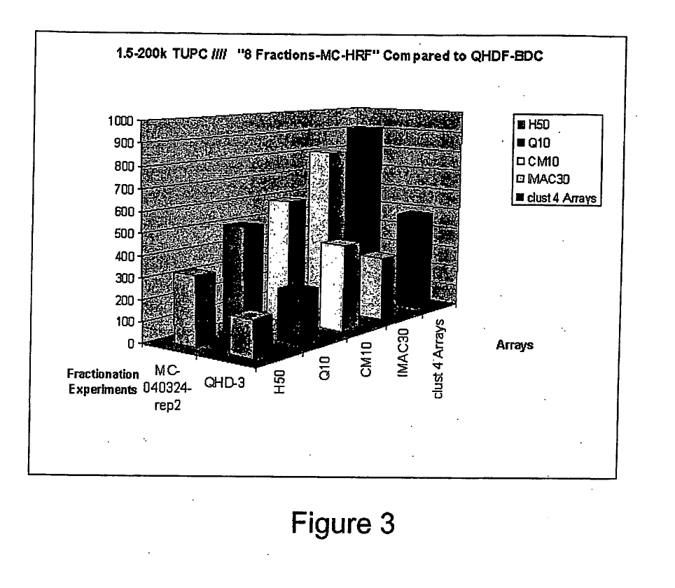

The Multiple chemistry fractionation method of the invention allows almost the doubling the number of unique peaks (clustered 4-arrays) as well as the total number of peaks (sum of 4-arrays) when compared to the standard fractionation on Q-HYPERD (See Table 5 and FIG. 3).

TABLE 5

| | Standard Q-HyperD | Invention |
|---|---|---|
| Number of Fractions | 6 | 8 |
| Separation Time (Days) | 0.5 | 1 |
| Total Number of Unique Peaks¹ | 480 | 905 (+89%) |
| Total Number of Peaks (4 Arrays) | 1,129 | 2,218 (+96%) |

¹(Cluster of 4 Arrays.)

### Example 2

### Multiple Chemistry Fractionation of Bovine Insulin-Spiked Human Serum on Mini-Columns

Each disposable WIZARD column was filled with 125 μL of one of the seven different sorbents as follows: Protein A (1 unit), Blue Trisacryl (3 units), Heparin (1 unit), MEP (1 unit), Green 5 (1 unit), and phenylpropylamine (2 units). The stack of 10 units was equilibrated with 3 ml of binding buffer (PBS (16v)/1 M Tris-HCl pH8 (9v)/H₂O (75v)) at a flow rate of 0.2 ml/min using a peristaltic pump. The flow was reduced to 0.01 ml/min for the sample injection. At the top of the Protein A first unit, 166 μL of human serum (bovine insulin-spiked or straight) five-fold diluted in 0.1 M Tris-HCl pH8 buffer. The first 1.25 mL collection at the bottom of the column-stack was discarded, and the next 1.25 mL effluent was collected as the flow-though fraction. Then the 10-column units were disconnected and all the sorbent contents were ejected from the columns in 1.5 mL micro-tubes by using 0.5 mL of the following eluents: TFA (0.4v)/H₂O (39.6v)/ACN (3.3v)/IPA (6.7v) for Protein A, Mep, and phenylpropylamine sorbents; and H40H (4v)/H₂O (36v)/ACN (3.3v)/IPA (6.7v) for the Blue Trisacryl, Heparin, Green 5 and Zirconia sorbents. The complete elution was performed by gentle mixing of the micro-tubes containing the mixtures of sorbent and eluents for one hour. The supernatants were recovered by slow centrifugation and pooled when coming from the same chemistry sorbent (Blue Trisacryl or phenylpropylamine). Samples of 300 μL of each of the 7 eluents corresponding to the seven different chemistries were frozen, lyophilized and then re-dissolved in 100 μl of 25 mM Tris-HCl pH 7.5 before analysis.

Lower Redundancy in the Fractions Distribution of Bovine Insulin Spiked in Human Serum

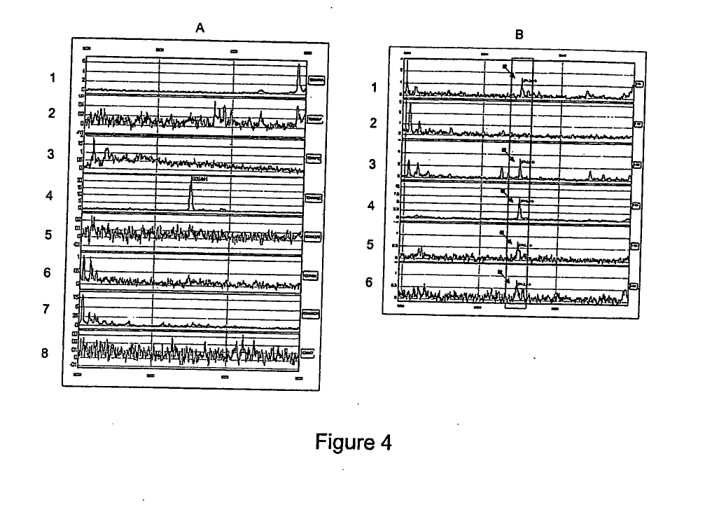

FIG. 4 illustrates the benefit of the method of the invention. Using the method of the invention, a sample spiked with insulin was detected on a specific sorbent chemistry (MEP-HYPERCEL, column A). In contrast, using prior art methods, represented by the anion exchange fractionation plate described in Example 1, insulin was detected in most of elution fractions from Q-HYPER-D with an undesirable signal dilution due to this spreading (column B).

Higher Sensitivity Conferred by Multiple Fractionation for Bovine Insulin Spiked in Human Serum

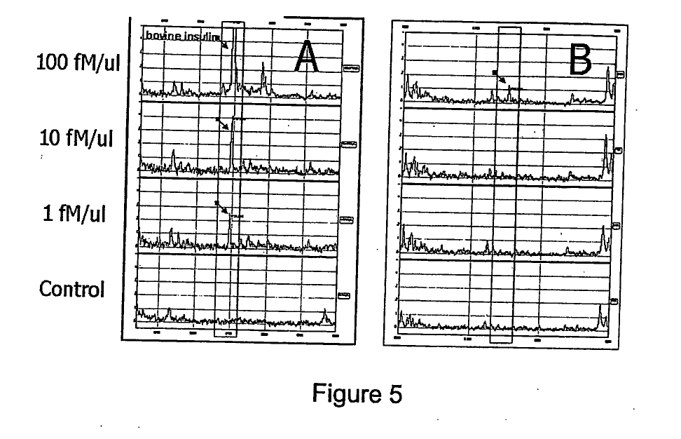

FIG. 5 shows the direct benefit on sensitivity provided by the method of invention. The ability of the method of the invention to capture insulin on a specific sorbent chemistry provides detection at concentrations as low as 1 fMol/μL in human serum (column A). Using prior art, single-chemistry fractionation methods (Q-HyperD), a 2-log reduction in sensitivity was observed (100 fMol/μL, column B). Thus, the method of the invention provides a marked improvement in the detection and identification of proteins or other biomolecular species of low-abundance.

### Example 3

### Separation of Human Serum Proteins by Their Hydrophobicity Degree

Three aliphatic hydrophobic supports with C2, C4, C8 hydrocarbon chains comprising primary amines as ligands are packed in three different Promega columns (125 μL of sorbent per column).

These hydrophobic sorbents are able to form hydrophobic association with proteins in physiological conditions of ionic strength and pH as a result of their unique chemical structure (see international patent application No. PCT/US2005/001304, which is hereby incorporated by reference). This property is very useful for this example since the buffer used for protein interaction is the same for all selected sorbents and do not comprise lyotropic agents as is generally the case for hydrophobic chromatography.

Columns were equilibrated with a physiological phosphate buffered saline (10 mM phosphate buffer, pH 7.2 containing 150 mM sodium chloride) and arranged in series, that is, the outlet of the first column is connected with the inlet of the second column and so on. 200 μL of albumin-depleted serum (protein concentration: 5 mg/mL) were introduced to the series of sobents. The sample was then pushed through the sectional columns using the initial physiological solution of phosphate buffered saline until absence of UV absorbance in the flowthrough.

The columns were then separated, and from each protein adsorbed were eluted using a mixture of TFA/ACN/IPA/Water (0.8%-6.7%-13.4%-79.2%). Collected proteins were then analyzed by mono-dimensional electrophoresis and SELDI MS.

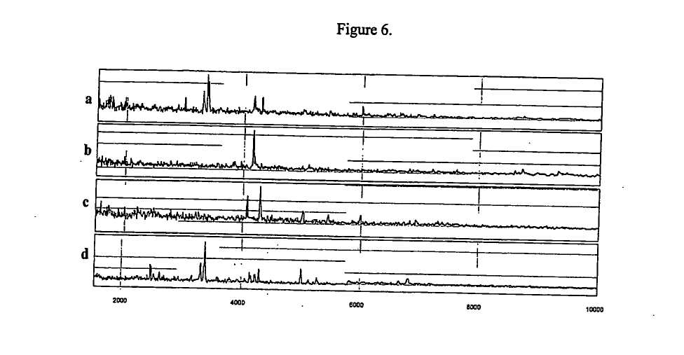

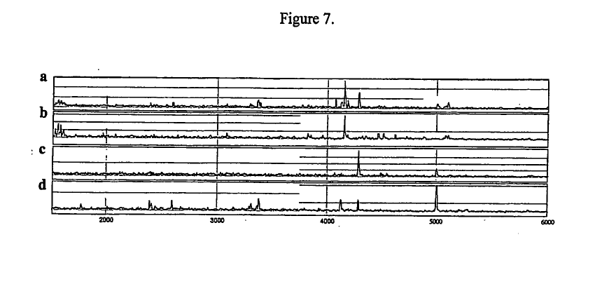

FIGS. 6 and 7 demonstrate that each sorbent captures different protein. Most of proteins of different category were sequentially captured by C2 and C4 sorbents. The C8 column adsorbed unique species previously uncaptured by the prior sorbents.

### Example 4

### Separation of Human Serum Proteins by Their Hydrophobicity Degree

While the previous experiment demonstrated the effectiveness of the separation principle, the first two columns adsorbed a large portion of the proteins in the sample.

To achieve a better fractionation of proteins based on hydrophobicity, a different series of aliphatic chain sorbent was used: C1, C2, C3, C4, and C6. As before, the ligands of these sorbents comprised primary amines. See international patent application No. PCT/US2005/001304.

C1 has a narrow specificity for hydrophobic associations and, therefore, interacts with the most hydrophobic species. Conversely the most hydrophobic sectional column (C6) has a large specificity for hydrophobic associations and, therefore, is expected to adsorb all proteins that escaped capture by previous columns, including those proteins with a weak property to form hydrophobic associations.

The series of HIC sorbents are evaluated in separate experiments using two different buffers. In one instance, the same conditions described in the previous example are used, and in a second a physiological buffer containing 2M urea is used. The latter buffer is used to slightly reduce the hydrophobic interaction of proteins for the sorbents.

After sample loading and washing, columns are separated and eluted as per the previous example. Collected proteins are then analyzed by mono-dimensional electrophoresis (SDS-PAGE) and SELDI MS. Analytical data show that proteins adsorbed and eluted from different sectional columns are different in their electrophoresis mobility and have a different molecular mass.

In the first experiment (absence of urea), proteins are located within the first part of the sorbent series (C1 to C3). In the second experiment (with urea), the proteins are moved downward to following hydrophobic columns.

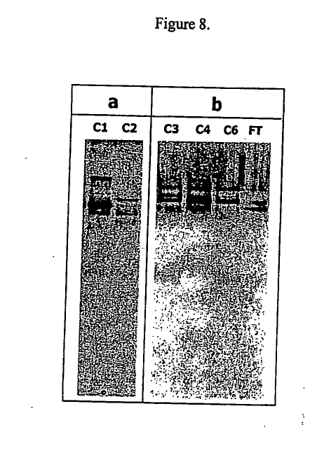

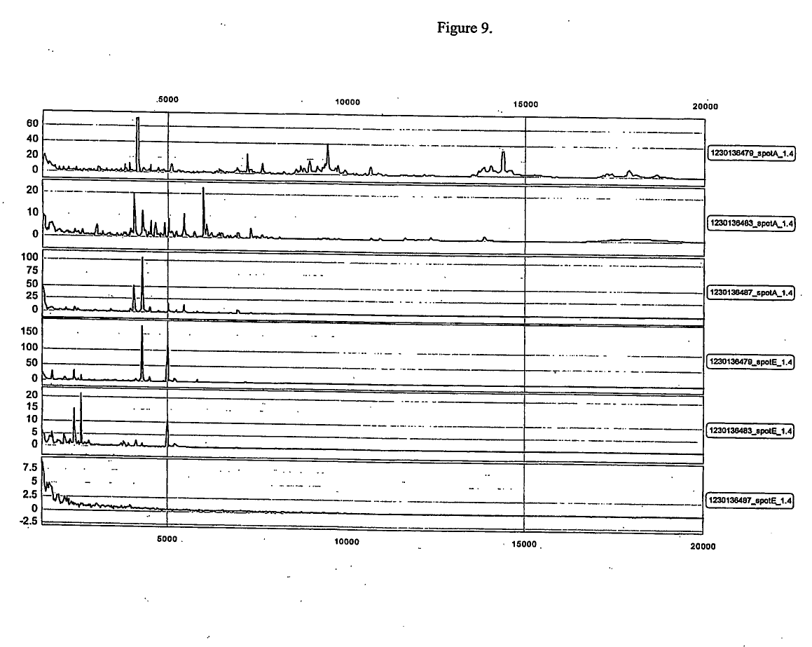

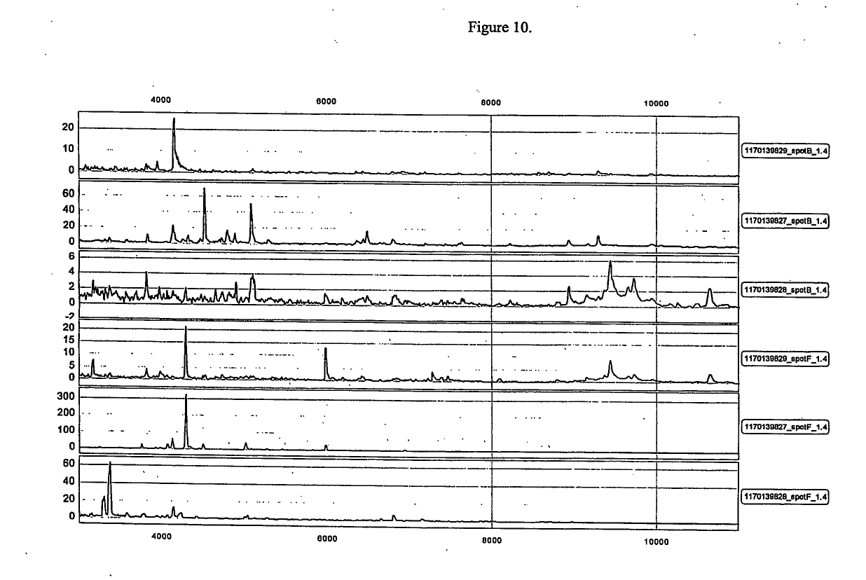

Regarding the experiment using urea, FIG. 8 shows that proteins adsorbed in the presence of urea 2 M and eluted from different sectional columns possess different electrophoresis mobilities and masses. FIG. 9 provides SELDI MS analysis of protein fractions eluted from C1, C2, C3, C4, C6 and FT (flowthrough), using a Q10 ProteinChip Array using a physiological buffer containing 2M urea. Similary, FIG. 10 provides SELDI MS analysis of protein fractions eluted from C1, C2, C3, C4, C6 and FT (flowthrough), using a CM10 ProteinChip Array using a physiological buffer containing 2M urea.

Thus, the present invention provides methods, apparatus, and kits for fractionating or prefractionating complex mixtures of biomolecular components. The methods, apparatus, and kits provided by the present invention provide means for detecting biomolecular components with greater sensitivity and ease that heretofore possible, thus providing better research and diagnostic tools among many other applications. It will be further appreciated that other examples of the many of the materials described herein can be used as described herein without departing from the spirit of scope of the invention. In particular, any material effective as a sorbent for biomolecular components or any method of detecting and identifying such component can be used as described herein.

1. A method comprising:
a. providing a series of at least three different sorbents arranged in a progression of decreasing specificity;
b. introducing a complex mixture to said series of sorbents;
c. contacting serially said complex mixture with each of said sorbents; and
d. capturing biomolecular components from said complex mixture on said sorbents, wherein each of said sorbents captures a substantially unique subset of said plurality of biomolecular components.

2. The method of claim 1, wherein said sorbents have specificities selected from the group consisting of high specificity, moderate specificity, and low specificity.

3. The method of claim 1, wherein at least one of said sorbents is a high specificity sorbent.

4. The method of claim 1, wherein at least one of said sorbents is a medium specificity sorbent.

5. The method of claim 1, wherein at least one of said sorbents is a low specificity sorbent.

6. The method of claim 1, wherein said series of sorbents comprises at least one high specificity sorbent, at least one medium specificity sorbent and at least one low specificity sorbent.

7. The method of claim 1, wherein all of said sorbents in said series are either high specificity sorbents, medium specificity sorbents or low specificity sorbents.

8. The method of claim 1, wherein at least two of said sorbents have the same degree of specificity.

9. The method of claim 1, wherein said contacting serially occurs as a continuous process.

10. The method of claim 1, further comprising selecting said sorbents to effect substantially complete removal of all biomolecular components from said complex mixture.

11. The method of claim 1, further comprising eluting said biomolecular components from at least one of said sorbents.

12. The method of claim 11, wherein said eluting includes exposing said at least one sorbent to water, a chaotropic agent, a lyotropic agent, an organic solvent, a change in ionic strength, a change in pH, a change temperature, a change pressure, or a combination of thereof.

13. The method of claim 12, further comprising subjecting said eluted biomolecular components to a second separation procedure.

14. The method according to claim 10, further comprising detecting at least one captured biomolecular component.

15. The method of claim 14, wherein said detecting includes detection using a method selected from the group consisting of: mass spectrometry, mono- and multi-dimensional gel electrophoresis, fluorimetric methods, high-pressure liquid chromatography, medium-pressure liquid chromatography.

16. The method of claim 15, further comprising determining the chemical identity of said detected biomolecular component.

17. The method of claim 16, further comprising capturing said mixture component on an adsorbent surface of a SELDI probe and determining the chemical identity of said mixture component by laser desorption-ionization mass spectrometry.

18. The method of claim 1, further comprising arranging said sorbents to form a substantially contiguous component-sequestering body.

19. The method of claim 18, further comprising arranging said sorbents in a substantially linear progression of adsorption specificities for at least one of said component types.

20. The method of claim 1, wherein each of said sorbents is a hydrophobic sorbent comprising a hydrocarbon chain and an amine ligand and wherein the hydrocarbon chain of each sorbent in the series comprises more carbons than that of the previous sorbent.

21. The method of claim 20, wherein said sorbents comprise hydrocarbon chains selected from the group consisting of C1, C2, C3, C4, C5 and C6.

22. A method comprising: contacting sequentially a complex mixture with (a) a biospecific adsorbent material, (b) a mixed-mode adsorbent material, and (c) a non-specific adsorbent material to capture thereby a plurality of biomolecular components from said complex mixture.

23. The method of claim 20, further comprising eluting said biomolecular components from at least one of said series of materials.

24. The method of claim 23, further comprising subjecting said eluted biomolecular components to a second separation procedure.

25. The method according to claim 23, further comprising detecting at least one captured biomolecular component.

26. The method of claim 25, wherein said detecting includes detection using a method selected from the group consisting of: mass spectrometry, mono- and multi-dimensional gel electrophoresis, fluorimetry, high-pressure liquid chromatography, medium-pressure liquid chromatography.

27. The method of claim 26, further comprising determining the chemical identity of said detected biomolecular component.

28. The method of claim 27, further comprising capturing said mixture component on an adsorbent surface of a SELDI probe and determining the chemical identity of said mixture component by laser desorption-ionization mass spectrometry.

29. The method of claim 20, further comprising eluting said mixture components from at least one of said materials.

30. A method comprising: contacting a complex mixture with a biospecific adsorbent material to reduce thereby the dynamic range of said complex mixture by at least a factor of 10 to provide thereby a low-abundance complex mixture; and contacting said low-abundance complex mixture with, in sequence, a mixed-mode adsorbent material and a non-specific adsorbent material to capture thereby substantially all of said plurality of biomolecular components from said complex mixture, wherein each of said materials captures a substantially unique subset of said plurality of biomolecular components.

31. The method of claim 30, further comprising eluting said biomolecular components from at least one of said adsorbent materials.

32. The method of claim 31, further comprising subjecting said eluted biomolecular components to a second separation procedure.

33. The method according to claim 31, further comprising detecting at least one captured biomolecular component.

34. The method of claim 33, wherein said detecting includes detection using a method selected from the group consisting of: mass spectrometry, mono- and multi-dimensional gel electrophoresis, fluorimetry, high-pressure liquid chromatography, medium-pressure liquid chromatography.

35. The method of claim 34, further comprising determining the chemical identity of said detected biomolecular component.

36. The method of claim 35, further comprising capturing said mixture component on an adsorbent surface of a SELDI probe and determining the chemical identity of said mixture component by laser desorption-ionization mass spectrometry.

37. An apparatus comprising: at least three sorbents characterized by different adsorption specificities for different biomolecular component types coupled in a serial arrangement of decreasing specificity.

38. The apparatus of claim 37, wherein said sorbents are arranged to define a progression in affinities for at least one biomolecular component type.

39. The apparatus of claim 38, wherein said apparatus defines a substantially contiguous component-sequestering body.

40. The apparatus of claim 39, wherein aid apparatus defines a substantially linear progression of adsorption specificities for at least one of said biomolecular component types.

41. The apparatus of claim 40, wherein said apparatus is columnar.

42. The apparatus of claim 40, wherein said apparatus defines an array of columns.

43. The apparatus of claim 37, wherein said apparatus defines a substantially linear progression of adsorption specificities for at least one of said biomolecular component types.

44. The apparatus of claim 43, wherein said apparatus is columnar.

45. The apparatus of claim 44, wherein said apparatus defines an array of columns.

46. The apparatus of claim 45, wherein said apparatus is provided in a stacked multi-well filtration plate format.

47. An apparatus comprising in sequence: (a) a high specificity sorbent, (b) a moderate specificity sorbent, and (c) a low specificity sorbent, and said sorbents being coupled in a serial arrangement whereupon introduction and passage of a buffered solution including (i) a complex mixture and (ii) a buffer that is compatible with said materials serially through said serial arrangement of said materials is effective to remove substantially all of said biomolecular components from said complex mixture.

48. The apparatus of claim 47, wherein said materials are arranged to define a progression in affinities for at least one biomolecular component type.

49. The apparatus of claim 48, wherein said apparatus defines a substantially contiguous component-sequestering body.

50. The apparatus of claim 49, wherein aid apparatus defines a substantially linear progression of adsorption specificities for at least one of said biomolecular component types.

51. The apparatus of claim 50, wherein said apparatus is columnar.

52. The apparatus of claim 50, wherein said apparatus defines an array of columns.

53. The apparatus of claim 47, wherein aid apparatus defines a substantially linear progression of adsorption specificities for at least one of said biomolecular component types.

54. The apparatus of claim 53, wherein said apparatus is columnar.

55. The apparatus of claim 54, wherein said apparatus defines an array of columns.

56. The apparatus of claim 55, wherein said apparatus is provided in a stacked plate format.

57. An kit comprising: at least three sorbents characterized by different adsorption specificities for different biomolecular components in a sample and a buffer compatible with the sorbents.

58. The kit of claim 57, wherein said sorbents are arranged to define a progression in affinities for at least one biomolecular component type.

59. The kit of claim 57, further including an elution buffer that is effective to elute said captured biomolecular components from said sorbents.

60. The kit of claim 59, further including an elution buffer that is effective to elute said captured biomolecular components from said sorbents.

61. The kit of claim 57, wherein said sorbents interact with biomolecular components based upon technologies selected from the group consisting of ion exchange, hydrophobic interaction chromatography, affinity chromatography and immunoaffinity.

62. The kit of claim 57, wherein said sorbents are selected from the group consisting of Protein A, Blue Trisacryl, Heparin, Mep, Green 5, Zirconia and phenylpropylamine cellulose.

63. A kit comprising: (a) a high specificity sorbent, (b) a moderate specificity sorbent, and (c) a low specificity sorbent, said materials being characterized by different adsorption specificities for different biomolecular component types and a compatible buffer.

64. The kit of claim 63, wherein said sorbents are arranged to define a progression in affinities for at least one biomolecular component type.

65. The kit of claim 63, further including an elution buffer that is effective to elute said captured biomolecular components from said sorbents.

66. The kit of claim 63, further including an elution buffer that is effective to elute said captured biomolecular components from said sorbents.

67. An apparatus comprising at least three detachable segments wherein each segment comprises a sorbent having a different adsorption specificity and wherein said segments are arranged in a progression of decreasing specificity of the sorbents.

68. The apparatus of claim 67, wherein said apparatus is columnar.

69. The apparatus of claim 67, wherein said apparatus defines an array of columns.

70. The apparatus of claim 67, wherein said apparatus is provided in a stacked multi-well filtration plate format.
